(19) United States
(12) Patent Application Publication
(10) Pub. No.: US 2023/0215573 A1
(43) Pub. Date: Jul. 6, 2023

(54) EVENT INFORMATION EVALUATION DEVICE, BIOLOGICAL INFORMATION EXTRACTION SYSTEM, EVENT INFORMATION EVALUATION SYSTEM AND BIOLOGICAL INFORMATION EXTRACTION DEVICE

(71) Applicant: PANASONIC INTELLECTUAL PROPERTY MANAGEMENT CO., LTD., Osaka (JP)

(72) Inventors: Matthew John LAWRENSON, Chesterfield, MO (US); Christopher John WRIGHT, London (GB); David Michael DUFFY, Zurich (CH); Bernadette ELLIOTT-BOWMAN, London (GB); Takeshi YOSHIHARA, Fukuoka (JP); Tsuyoshi NAKAMURA, Fukuoka (JP); Yasunori WATANABE, Fukuoka (JP); Kota MORISAKI, Fukuoka (JP)

(73) Assignee: PANASONIC INTELLECTUAL PROPERTY MANAGEMENT CO., LTD., Osaka (JP)

(21) Appl. No.: 17/927,803

(22) PCT Filed: May 19, 2021
(86) PCT No.: PCT/JP2021/019076

§ 371 Date: Nov. 25, 2022

(30) Foreign Application Priority Data
May 25, 2020 (JP) 2020-090695

(51) Int. Cl. G16H 50/30; G16H 10/60

(52) U.S. Cl. CPC G16H 50/30; G16H 10/60

(57) ABSTRACT

A biological information extraction device is capable of communicating with a plurality of terminals. Each of the plurality of terminals is configured to acquire position information of a user and biological information of the user. The biological information extraction device includes: a processor; and a memory storing instructions that, when executed by the processor, cause the biological information extraction device to perform operations. The operations include: acquiring the position information and the biological information from at least one of the plurality of terminals; accumulating a number of users having a same value of the biological information corresponding to the position information, and selecting biological information corresponding to a largest accumulation number of users for each piece of the position information.

### TECHNICAL FIELD

The present disclosure relates to an event information evaluation device, a biological information extraction system, an event information evaluation system, and a biological information extraction device that associate biological information corresponding to predetermined position information with event information.

### BACKGROUND ART

In recent years, smartphones, mobile phones, and tablet terminals are widely used, and anyone can easily capture images using a camera function thereof. In addition, there are many users who upload the captured images to a content such as a social network service (SNS). There are many contents in which position information is added to an image uploaded by the user.

In the field of analyzing biological information, biological information is acquired from a heart rate and a perspiration amount of a user using a wearable terminal, and emotion information is estimated.

In this way, in recent years, a situation in which position information and emotion information can be easily acquired has been prepared. Patent Literature 1 discloses a technique of a communication system that guides an action for causing a predetermined feeling to a user in accordance with current position information of a user and map information to which emotion information of a user is mapped.

### CITATION LIST

### Patent Literature

Patent Literature 1: WO2015/162949A1

### SUMMARY OF INVENTION

### Technical Problem

Although the technique of Patent Literature 1 discloses the communication system that generates guidance information in accordance with the current position information of a user and the map information to which the emotion data is mapped, the number of users at each location and an indication of an emotional state of the majority of users at each location are not disclosed. Therefore, a content provider at each position cannot cumulatively confirm reaction or emotion of the user from the biological information of the user with respect to the provided content. As a result, information for the content provider to improve the content is reduced, which makes it difficult to improve the content.

The present disclosure has been devised in view of the circumstances in the related art described above, and an object thereof is to provide an event information evaluation device, a biological information extraction system, an event information evaluation system, and a biological information extraction device in which position information of a user and biological information of the user are acquired, the number of users for is accumulated each position, and biological information corresponding to a largest accumulation number of users is selected for each position, so that reaction of the users with respect to a content at the position is confirmed from the biological information of the users.

### Solution to Problem

In view of the above-described situation, the present disclosure provides a biological information system including a plurality of terminals and an information control device. The terminal includes: a position information acquisition unit configured to acquire position information of a user; a biological information extraction unit configured to acquire biological information of the user; and a terminal information communication unit configured to communicate the position information and the biological information to outside. The information control device includes: a center information communication unit configured to communicate with the terminal information communication unit and receive the position information and the biological information; an accumulation unit configured to accumulate a number of users having a same value of biological information for each position based on the position information and the biological information received by the center information communication unit; and an accumulation information selection unit configured to select biological information corresponding to a largest accumulation number of users for each piece of the position information. Consequently, it is possible to confirm reaction of the users with respect to a content in the position.

In addition, the present disclosure provides an event information evaluation system including a plurality of terminals. The terminal includes: a position information acquisition unit configured to acquire position information of a user; a biological information extraction unit configured to acquire biological information of the user; and a terminal information communication unit configured to communicate the position information and the biological information to outside. The event information evaluation system further includes: a center information communication unit configured to communicate with the terminal information communication unit and receive the position information and the biological information; an accumulation unit configured to accumulate a number of users having a same value of biological information for each position based on the position information and the biological information received by the center information communication unit; and an event information providing unit configured to provide event information to the user. Biological information corresponding to a largest accumulation number of users for each piece of position information is selected and the event information is associated with the biological information corresponding to the largest accumulation number of users, so that reaction of the users with respect to a content at the position can be confirmed from the biological information of the users.

In addition, the present disclosure provides a biological information extraction device including: a center information communication unit configured to communicate with a terminal information communication unit configured to communicate position information of a user and biological information of a user to outside, and receive the position information and the biological information; an accumulation unit configured to accumulate a number of users having a same value of biological information for each position based on the position information and the biological information received by the center information communication unit; and an accumulation information selection unit configured to select biological information corresponding to a largest accumulation number of users for each piece of the position information. Therefore, reaction of the users with respect to a content in the position can be confirmed from the biological information of the users

In addition, the present disclosure provides an event information evaluation device including: a center information communication unit configured to communicate with a terminal information communication unit configured to communicate position information of a user and biological information of a user to outside, and receive the position information and the biological information; an accumulation unit configured to accumulate, based on the position information received by the center information communication unit, a number of users having a same value of the biological information for the position; an event information providing unit configured to provide event information to a user. Biological information corresponding to a largest accumulation number of users for each piece of position information is selected and the event information is associated with the biological information corresponding to the largest accumulation number of users, so that reaction of the users with respect to a content at the position can be confirmed from the biological information of the users.

### Advantageous Effects of Invention

According to the present disclosure, by acquiring position information of a user and biological information of the user, accumulating the number of users for each position, and selecting biological information corresponding to a largest accumulation number of users for each position, it is possible to confirm reaction of the users with respect to a content at the position from the biological information of the users.

### BRIEF DESCRIPTION OF DRAWINGS

FIG. 1 is a detailed configuration diagram of a biological information extraction system according to a first embodiment.

FIG. 2 is a diagram showing an operation flow of the biological information extraction system according to the first embodiment.

FIG. 3 is a diagram showing an example of a table in which position information of users and biological information of the users are aggregated in the biological information extraction system according to the first embodiment.

FIG. 4 is a diagram showing an example of a table in which biological information of users at each position is accumulated in the biological information extraction system according to the first embodiment.

FIG. 5 is a diagram showing an example of a table in which biological information corresponding to a largest accumulation number of users is extracted for each position in the biological information extraction system according to the first embodiment.

FIG. 6 is a diagram showing an example of display data generated based on the position information of the users, the biological information of the users, and map information in the biological information extraction system according to the first embodiment.

FIG. 7 is a diagram showing an example of display data generated based on the map information and the biological information corresponding to the largest accumulation number of users for each position in the biological information extraction system according to the first embodiment.

FIG. 8 is a detailed configuration diagram of a biological information extraction system according to a second embodiment.

FIG. 9 is a diagram showing an operation flow of the biological information extraction system according to the second embodiment. FIG. 10 is a diagram showing an example of a table in which position information of users and emotion information of the users are aggregated in the biological information extraction system according to the second embodiment.

FIG. 11 is a diagram showing an example of a table in which emotion information of users at each position is accumulated in the biological information extraction system according to the second embodiment.

FIG. 12 is a diagram showing an example of a table in which emotion information having a largest accumulation number of users is selected for each position in the biological information extraction system according to the second embodiment.

FIG. 13 is a diagram showing display data generated based on the position information of the users, the emotion information of the users, and map information in the biological information extraction system according to the second embodiment.

FIG. 14 is a diagram showing display data generated based on the map information and the emotion information having the largest accumulation number of users for each position in the biological information extraction system according to the second embodiment.

### DESCRIPTION OF EMBODIMENTS

Hereinafter, embodiments of an event information evaluation device, a biological information extraction system, an event information evaluation system, and a biological information extraction device according to the present disclosure will be described in detail with reference to the drawings as appropriate. An unnecessarily detailed description may be omitted. For example, a detailed description of a well-known matter or repeated descriptions of substantially the same configuration may be omitted. This is to avoid unnecessary redundancy in the following description and to facilitate understanding for those skilled in the art. It should be noted that the accompanying drawings and the following description are provided for a thorough understanding of the present disclosure by those skilled in the art, and are not intended to limit the subject matter recited in the claims.

Hereinafter, embodiments of the present invention will be described with reference to the drawings. In the following embodiments, a biological information extraction device constituting a biological information extraction system will be described as an example.

### First Embodiment

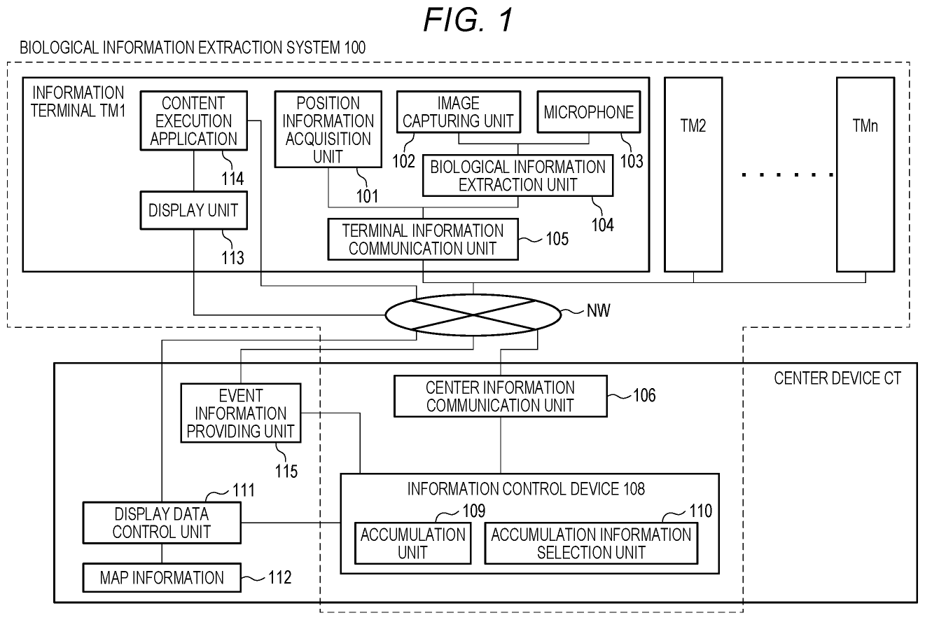

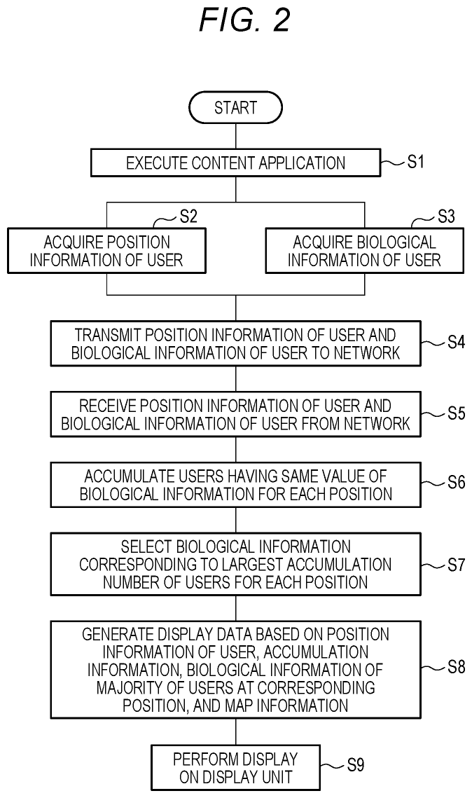

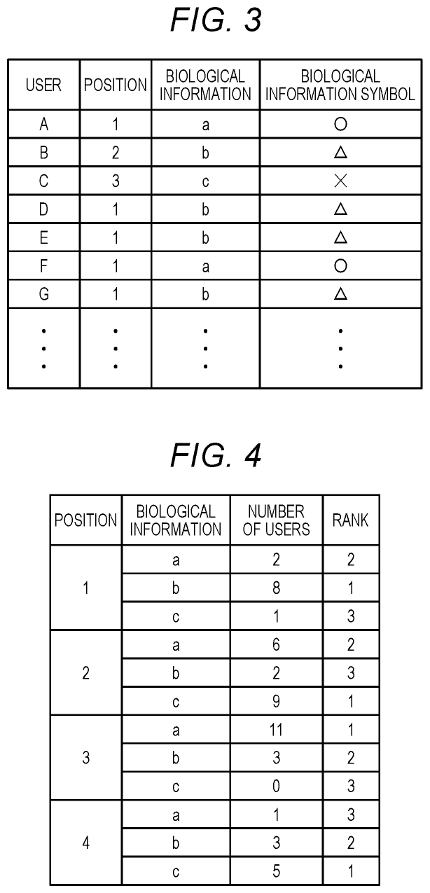

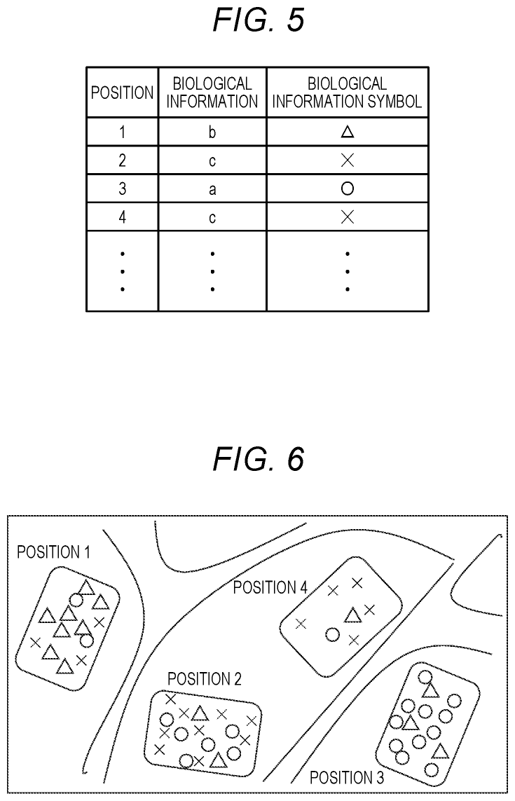

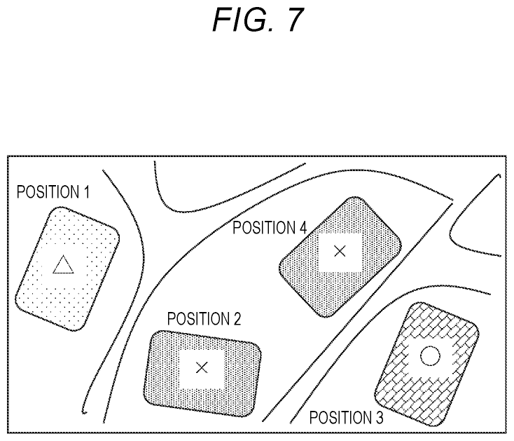

FIG. 1 is a detailed configuration diagram of a biological information extraction system 100 according to a first embodiment. FIG. 2 is a diagram showing an operation flow of the biological information extraction system 100 according to the first embodiment. FIG. 3 is a diagram showing an example of a table in which position information of users and biological information of the users are aggregated in the biological information extraction system 100 according to the first embodiment. FIG. 4 is a diagram showing an example of a table in which biological information of users at each position is accumulated in the biological information extraction system 100 according to the first embodiment. FIG. 5 is a diagram showing an example of a table in which biological information corresponding to a largest accumulation number of users is selected for each position in the biological information extraction system 100 according to the first embodiment. FIG. 6 is a diagram showing an example of display data generated based on the position information of the users, the biological information of the users, and map information in the biological information extraction system 100 according to the first embodiment. FIG. 7 is a diagram showing an example of display data generated based on the map information and the biological information corresponding to the largest accumulation number of users for each position in the biological information extraction system 100 according to the first embodiment.

As shown in FIG. 1, the biological information extraction system 100 includes a plurality of information terminals TM1, TM2, . . . , and TMn (hereinafter referred to as “information terminal TM” in the present description), a center information communication unit 106, an emotion information analysis unit 107, and an information control device 108. n is an integer of 2 or more. The information terminals TM each similarly include a position information acquisition unit 101, an image capturing unit 102, a microphone 103, a biological information extraction unit 104, a terminal information communication unit 105, a display unit 113, and a content execution application 114. The information control device 108 includes an accumulation unit 109 and an accumulation information selection unit 110. The center information communication unit 106 and the information control device 108 constitute a biological information extraction device according to the present disclosure. Each of the position information acquisition unit 101, the biological information extraction unit 104, the terminal information communication unit 105, the content execution application 114, the center information communication unit 106, the accumulation unit 109, and the accumulation information selection unit 110 is implemented by, for example, a processor such as a central processing unit (CPU), and is functionally achieved by the processor reading a program and data stored in advance in a memory (not shown). The processor may be implemented by a digital signal processor (DSP) or a field programmable gate array (FPGA).

The information terminals TM transmits and receives user information and analysis information by communicating with a center device CT, respectively. The information to be communicated is not limited to the user information and the analysis information. The information to be communicated may be content information or event information. The information terminal TM may be, for example, a smartphone or a tablet terminal, or may be implemented as a wearable microphone that can be worn by a person.

The position information acquisition unit 101 serving as an example of a processing device acquires position information of a user. A method for acquiring the position information of a user is a known technique disclosed in, for example, WO2019/187824A1, and therefore description thereof will be omitted.

The biological information extraction unit 104 serving as an example of the processing device extracts biological information of a user. Specifically, the biological information extraction unit 104 extracts heartbeat of a user based on video acquired by the image capturing unit 102. In addition, the biological information extraction unit 104 extracts vocal cord information based on voice of a user acquired by the microphone 103. Further, when the information terminal TM is a wearable terminal, the biological information extraction unit 104 may extract body temperature of a user and a perspiration state of a user. A method for extracting detailed biological information is, for example, a known technique disclosed in JP2017-153773A, and therefore description thereof will be omitted.

The terminal information communication unit 105 serving as an example of a processing device is an interface that transmits the position information of the user acquired from the position information acquisition unit 101 and the biological information of the user acquired from the biological information extraction unit 104 to the center device CT via network NW.

The center information communication unit 106 constituting the biological information extraction device and serving as an example of the processing device is an interface that receives the position information of the user and the biological information of the user which are transmitted by the terminal information communication unit 105 via the network NW.

The information control device 108 constituting the biological information extraction device and serving as an example of the processing device associates the position information of the user with the biological information of the user on based on the position information of the user and the biological information of the user received by the center information communication unit 106 (that is, association).

The accumulation unit 109 constituting the biological information extraction device and serving as an example of the processing device accumulates the number of users having the same value of the biological information for each position (specifically, for each position information) of the users based on the position information of each user and the biological information of each user. A range of the accumulated user position is not limited to the above, and may be determined for each facility or may be set by a user freely.

The accumulation information selection unit 110 constituting the biological information extraction device and serving as an example of the processing device selects biological information corresponding to a largest accumulation number of users for each position, based on the biological information indicating the same value for each user position accumulated by the accumulation unit 109. Note that information to be selected is not limited to the above, and a user may set a selection condition freely.

An event information evaluation device according to the present disclosure includes at least the center information communication unit 106, the information control device 108, and an event information providing unit 115. In addition, an event information evaluation system according to the present disclosure includes at least the plurality of information terminals TM and the information control device 108.

Next, an operation of associating the position information of the user with the biological information of the user in the biological information extraction system 100 of the first embodiment will be described with reference to FIG. 2.

In FIG. 2, the biological information extraction system 100 is started by a user executing the content execution application 114 in the information terminal TM (S1). The content execution application 114 is an application for executing (for example, playing or displaying) a content, and is provided from the event information providing unit 115 of the center device CT to the information terminal TM via the network NW. When the content execution application 114 is executed by the operation of the user, the position information acquisition unit 101 acquires current position information of the user (S2). In parallel with the acquisition of the position information of the user by the position information acquisition unit 101, the biological information extraction unit 104 acquires the video of the user from the image capturing unit 102, and acquires the voice of the user from the microphone 103. The biological information extraction unit 104 extracts biological information from the acquired video of the user and the voice of the user so as to acquire the biological information (S3). The terminal information communication unit 105 acquires the position information of the user and the biological information of the user from the biological information extraction unit 104. In addition, the terminal information communication unit 105 transmits the position information of the user and the biological information of the user to the center device CT via the network NW (S4).

The center information communication unit 106 receives the position information of the user and the biological information of the user via the network NW (S5). In the information control device 108 or the accumulation information selection unit 110, the position information of the user and the biological information of the user are associated with each other. The accumulation unit 109 aggregates position information of users and biological information of the users, and accumulates the number of users having the same value of the biological information for each position (S6). The accumulation information selection unit 110 selects biological information corresponding to the largest accumulation number of users for each position with respect to the biological information of the users for each position accumulated by the accumulation unit 109, and sets the selected biological information as biological information representing the position (S7). A display data control unit 111 creates display data in which the position information of the user, the accumulation information, and the biological information of the majority of users at the position transmitted from the information control device 108 are superimposed on a map read from map information 112 (S8). The display unit 113 of the information terminal TM receives the display data generated by the display data control unit 111 via the network NW and displays the generated display data (S9).

Next, an information control method in the information control device 108 will be described using a specific example with reference to FIGS. 3, 4, 5, 6, and 7.

In the specific example, positions to be evaluated are four positions including position 1, position 2, position 3, and position 4. Each of a, b, and c indicates, for example, the type of biological information such as the pulse rate, the tone of voice, and the body temperature. In addition, a, b, and c may indicate numerical values for the same type of biological information, respectively. In this case, for example, a, b, and c indicate body temperature 36° C., 37° C., and 38° C., respectively. The biological information is classified based on the biological information, and symbolized biological information symbols are represented by O, Δ, and X. For example, the body temperature is classified as O for 36.0° C. to 36.9° C., Δ for 37.0° C. to 37.9° C., and X for 38.0° C. to 38.9° C.

When the position information of the user and the biological information of the user are acquired from the center information communication unit 106, the accumulation unit 109 associates the position information of the user with the biological information of the user, and creates a table shown in FIG. 3. The accumulation unit 109 accumulates the number of users having the biological information of the same value (for example, biological information or biological information symbol, or both of them) at each position based on the table shown in FIG. 3, and creates a table shown in FIG. 4. The accumulation information selection unit 110 selects biological information corresponding to the largest accumulation number of users for each position based on the table shown in FIG. 4. In other words, the accumulation information selection unit 110 selects biological information whose rank is “1” for each position in FIG. 4. The accumulation information selection unit 110 generates a table shown in FIG. 5 based on the selected biological information.

The display data control unit 111 creates display data based on the tables shown in FIGS. 3 and 5 acquired from the information control device 108 and the map information 112 stored in advance. For example, FIG. 6 is display data generated by the display data control unit 111 based on the table of FIG. 3 and the map information. In the display data shown in FIG. 6, the position of each user and the biological information of each user are displayed. By generating the display data shown in FIG. 6, a user and a content provider can visually recognize a gathering state of the users and the biological information of the users on the map at the information terminal TM and the center device CT. The display data shown in FIG. 7 indicates the biological information of the majority of users at the position for each position. By generating the display data shown in FIG. 7, the user and the content provider can visually recognize the biological information for each position on the map at the information terminal TM and the center device CT. As a result, by generating the display data of FIGS. 6 and 7, the user and the content provider can easily evaluate the provided content.

Note that this specific example is merely an example, and the configuration is not limited thereto. For example, the biological information selected in FIG. 5 may be selected from any rank in FIG. 4. The event information sent from the event information providing unit 115 is not limited to the content information. For example, the information may be disaster information or event information.

### Second Embodiment

A second embodiment is a form in which the biological information in the first embodiment is further analyzed by an emotion information analyzing unit 107 to be described later to select the emotion information of the user.

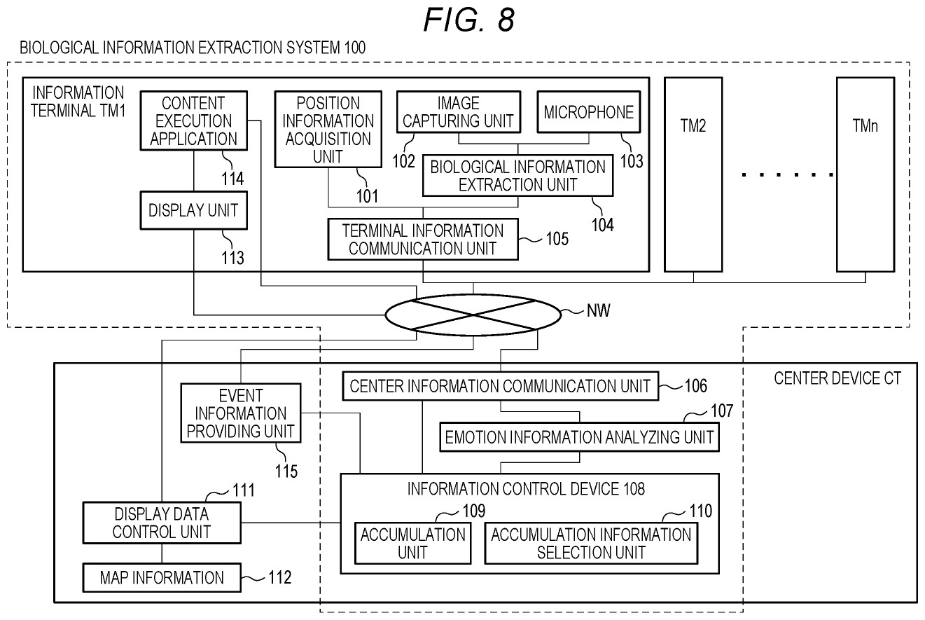

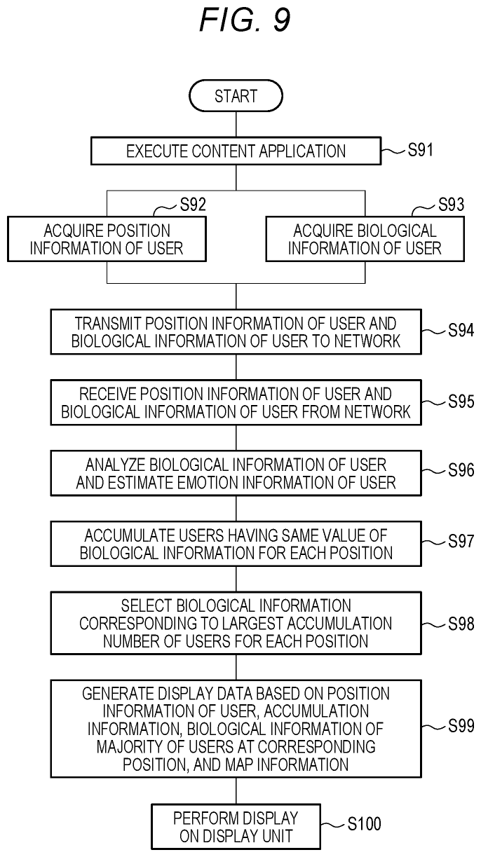

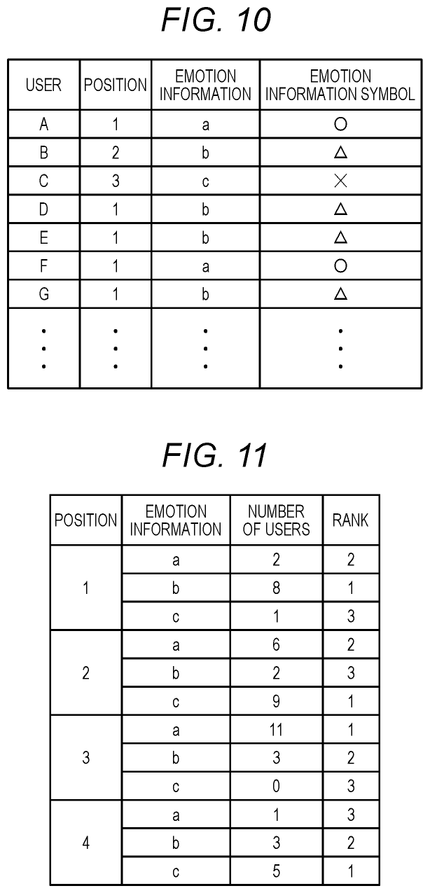

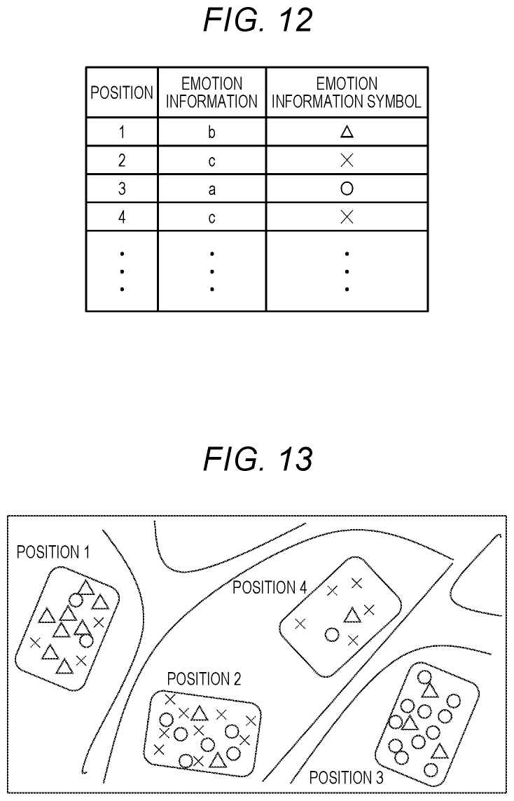

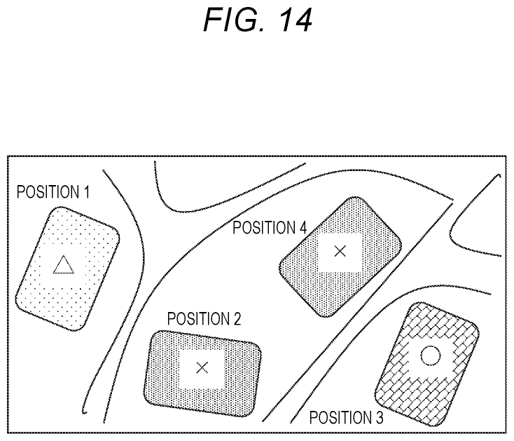

The second embodiment will be described with reference to FIG. 8. FIG. 8 is a detailed configuration diagram of the biological information extraction system 100 according to the second embodiment. FIG. 9 is a diagram showing an operation flow of the biological information extraction system 100 according to the second embodiment. FIG. 10 is a diagram showing an example of a table in which position information of users and emotion information of the users are aggregated in the biological information extraction system 100 according to the second embodiment. FIG. 11 is a diagram showing an example of a table in which emotion information of users at each position is accumulated in the biological information extraction system 100 according to the second embodiment. FIG. 12 is a diagram showing an example of a table in which emotion information having a largest accumulation number of users is selected for each position in the biological information extraction system according to the second embodiment. FIG. 13 is a diagram showing display data generated based on the position information of the users, the emotion information of the users, and map information in the biological information extraction system according to the second embodiment. FIG. 14 is a diagram showing display data generated based on the map information and the emotion information having the largest accumulation number of users for each position in the biological information extraction system according to the second embodiment. A difference between the second embodiment and the first embodiment is whether the emotion information analyzing unit 107 is provided. The same components as those of the first embodiment are denoted by the same reference numerals, and the description thereof will be simplified or omitted, and different contents will be described.

The biological information extraction system 100 according to the second embodiment includes the emotion information analyzing unit 107 in addition to the configuration of the biological information extraction system according to the first embodiment. The emotion information analyzing unit 107 is a part of the biological information extraction device.

The emotion information analyzing unit 107 constituting the biological information extraction device and serving as an example of the processing device analyzes the biological information of the users received by the center information communication unit 106, and estimates emotion information of the user based on an analysis result. A detailed emotion information estimating method is, for example, a known technique disclosed in JP2019-040525A, and therefore description thereof will be omitted.

Next, an operation of associating the position information of the user with the emotion information of the user in the biological information extraction system 100 of the second embodiment will be described with reference to FIG. 9.

In FIG. 9, the biological information extraction system 100 is started by executing the content execution application 114 by operation on the information terminal TM of the user (S91). The content execution application 114 is provided from the event information providing unit 115 of the center device CT to the information terminal TM via the network NW. When the content execution application 114 is executed by the operation of the user, the position information acquisition unit 101 acquires current position information of the user (S92). In parallel with the acquisition of the position information of the user by the position information acquisition unit 101, the biological information extraction unit 104 acquires the video of the user from the image capturing unit 102, and acquires the voice of the user from the microphone 103. The biological information extraction unit 104 extracts biological information from the acquired video of the user and the voice of the user so as to acquire the biological information (S93). The terminal information communication unit 105 acquires the position information of the user and the biological information of the user from the biological information extraction unit 104. In addition, the terminal information communication unit 105 transmits the position information of the user and the biological information of the user to the center device CT via the network NW (S94).

The center information communication unit 106 receives the position information of the user and the biological information of the user via the network NW (S95). The emotion information analyzing unit 107 acquires the biological information of the user received by the center information communication unit 106, and analyzes the biological information of the user. The emotion information analyzing unit 107 estimates the emotion information of the user based on an analysis result of the biological information of the user (S96). In the information control device 108 (for example, the accumulation information selection unit 110), the position information of the user and the emotion information of the user are associated with each other. The accumulation unit 109 aggregates position information of users and emotion information of the users, and accumulates the number of users having the same value of the emotion information for each position (S97). The accumulation information selection unit 110 selects emotion information having the largest accumulation number of users for each position with respect to the emotion information of the user for each position accumulated by the accumulation unit 109, and sets the selected emotion information as emotion information of the majority of users at the position (S98). The display data control unit 111 generates display data based on the position information of the user, the accumulation information, the emotion information of the majority of users at the position which are transmitted from the information control device 108, and the map information 112 (S99). The display unit 113 receives the display data generated by the display data control unit 111 via the network NW and displays the generated display data (S100).

Next, an information control method in the information control device 108 will be described using a specific example with reference to FIGS. 10, 11, 12, 13, and 14.

In the specific example, positions to be evaluated are four positions including position 1, position 2, position 3, and position 4. There are three types of emotional information a, b, and c to be evaluated. Each of a, b, and c indicates a type of emotional information such as a concentration degree and delight, anger, sorrow and pleasure. In addition, numerical values may be as indication for the same type of emotion information. In this case, for example, a, b, and c indicate concentration degrees 10, 50, and 100, respectively. The emotion information is classified based on the emotion information, and symbolized emotion information symbols are represented by O, Δ, and X. For example, the body temperature is classified as O for 36.0° C. to 36.9° C., Δ for 37.0° C. to 37.9° C., and X for 38.0° C. to 38.9° C.

The accumulation unit 109 acquires the position information of the user from the center information communication unit 106, and acquires the biological information of the user from the emotion information analyzing unit 107. The accumulation unit 109 associates the position information of the user with the emotion information of the user, so as to create a table shown in FIG. 10. The accumulation unit 109 accumulates the number of users for each emotion information at each position based on the table shown in FIG. 10, and creates a table shown in FIG. 11. The accumulation information selection unit 110 selects biological information corresponding to the largest accumulation number of users for each position based on the table shown in FIG. 11. In other words, the accumulation information selection unit 110 selects emotion information whose rank is “1” for each position in FIG. 11. The accumulation information selection unit 110 generates a table shown in FIG. 12 based on the selected emotion information.

The display data control unit 111 creates display data based on the tables shown in FIGS. 10 and 12 acquired from the information control device 108 and the map information 112 stored in advance. For example, FIG. 13 is display data generated by the display data control unit 111 based on the table of FIG. 10 and the map information. In the display data shown in FIG. 13, the position of each user and the emotion information of each user are displayed. By generating the display data shown in FIG. 13, a user and a content provider can visually recognize a gathering state of the users and the emotion information of the users on the map at the information terminal TM and the center device CT. The display data shown in FIG. 14 indicates emotion information of the majority of users at the position for each position. By generating the display data shown in FIG. 14, the user and the content provider can visually recognize the emotion information for each position on the map at the information terminal TM and the center device CT. As a result, by generating the display data of FIGS. 13 and 14, the user and the content provider can easily evaluate the provided content.

Note that this specific example is merely an example, and the configuration is not limited thereto. For example, the biological information selected in FIG. 12 may be selected from any rank in FIG. 11. The event information sent from the event information providing unit 115 is not limited to the content information. For example, the information may be disaster information or event information.

Finally, configurations, operations, and effects of the biological information extraction system and the biological information extraction device according to the present disclosure will be described.

An embodiment according to the present disclosure provides an event information evaluation device including: a center information communication unit configured to communicate with a terminal information communication unit configured to communicate position information of a user and biological information of a user to outside, and receive the position information and the biological information; an accumulation unit configured to accumulate, based on the position information received by the center information communication unit, the number of users having a same value of the biological information corresponding to the position information; an event information providing unit configured to provide event information to a user; and an accumulation information selection unit configured to select biological information corresponding to a largest accumulation number of users for each piece of the position information and associate the event information with the biological information corresponding to the largest accumulation number of users.

In this configuration, the event information evaluation device acquires the position information of the user and the biological information of the user, accumulates the biological information of the users for each position of the user, selects the biological information corresponding to the largest accumulation number of users for each position, and associates the biological information corresponding to the largest accumulation number of users with the event information.

Accordingly, the event information evaluation device can cause the content provider to confirm information in which the biological information of the user and the event information are associated with each other. As a result, the content provider can evaluate the content.

In an embodiment of the present disclosure, the event information evaluation device further includes an emotion information analyzing unit configured to analyze the biological information and estimate emotion information of a user.

In this configuration, the event information evaluation device further analyzes the biological information and estimates the emotion information of the user in the information control device.

Accordingly, the event information evaluation device can allow the content provider to acquire more detailed information on a state of the user. As a result, the content provider can evaluate the content in more detail.

An embodiment according to the present disclosure provides a biological information extraction system including: a plurality of terminals; and an information control device, in which the terminals each include a position information acquisition unit configured to acquire position information of a user, a biological information extraction unit configured to acquire biological information of a user, and a terminal information communication unit configured to communicate the position information and the biological information to outside, and the information control device includes a center information communication unit configured to communicate with the terminal information communication unit and receive the position information and the biological information, an accumulation unit configured to accumulate, based on the position information received by the center information communication unit, the number of users having a same value of the biological information of the position, and an accumulation information selection unit configured to select biological information corresponding to a largest accumulation number of users for each position.

In this configuration, the biological information extraction system acquires the position information of the user and the biological information of the user, accumulates the biological information of the users for each position of the user, and selects the biological information corresponding to the largest accumulation number of users for each position.

Accordingly, the biological information extraction system can allow the content provider to easily confirm the biological information of the users for each position.

In an embodiment of the present disclosure, the biological information corresponding to the largest accumulation number of users is biological information of majority of the users for each position.

In this configuration, the biological information extraction system sets biological information corresponding to the largest accumulation number of users as the biological information of majority of the users for each position.

Accordingly, the biological information extraction system can allow the content provider to easily confirm the biological information of the largest number of users for each position.

In an embodiment of the present disclosure, the biological information extraction system further includes an emotion information analyzing unit configured to analyze the biological information and estimate emotion information of a user.

In this configuration, the biological information extraction system analyzes the biological information of the user and estimates the emotion information of the user.

Accordingly, the biological information extraction system can acquire more detailed information about the state of the user.

In an embodiment of the present disclosure, the biological information extraction system further includes a display data control unit configured to acquire map information and generate display data in which biological information of majority of the users and the map information are associated with each other.

In this configuration, the biological information extraction system acquires the map information and generates the display data in which the biological information of the user and the map information are associated with each other.

Accordingly, the biological information extraction system can display the biological information of each user on the map. As a result, the content provider can easily visually recognize the biological information of the user and the position information of the user.

An embodiment according to the present disclosure provides an event information evaluation system including: a plurality of terminals each including a position information acquisition unit configured to acquire position information of a user, a biological information extraction unit configured to acquire biological information of a user, and a terminal information communication unit configured to communicate the position information and the biological information to outside; a center information communication unit configured to communicate with the terminal information communication unit and receive the position information and the biological information; an accumulation unit configured to accumulate, based on the position information received by the center information communication unit, the number of users having a same value of the biological information of the position; an event information providing unit configured to provide event information to a user; and an accumulation information selection unit configured to select biological information corresponding to a largest accumulation number of users for each position and associate the event information with the biological information corresponding to the largest accumulation number of users.

In this configuration, the event information evaluation system acquires the position information of the user and the biological information of the user, accumulates the biological information of the users for each position of the user, selects the biological information corresponding to the largest accumulation number of users for each position, and associates the biological information corresponding to the largest accumulation number of users with the event information.

Accordingly, the event information evaluation system can cause the content provider to confirm information in which the biological information of the user and the event information are associated with each other. As a result, the content provider can evaluate the content.

In an embodiment of the present disclosure, the biological information corresponding to the largest accumulation number of users is biological information of majority of the users for each position.

In this configuration, the event information evaluation system sets the biological information corresponding to the largest accumulation number of users as the biological information of majority of the users for each position.

Accordingly, the event information evaluation system can allow the content provider to easily confirm the biological information of the majority of users for each position. As a result, the content provider can effectively evaluate the content.

In an embodiment of the present disclosure, the event information evaluation system further includes an emotion information analyzing unit configured to analyze the biological information and estimate emotion information of a user.

In this configuration, the event information evaluation system further analyzes the biological information and estimates the emotion information of the user in the information control device.

Accordingly, the event information evaluation system can allow the content provider to acquire more detailed information on a state of the user. As a result, the content provider can evaluate the content in more detail.

An embodiment according to the present disclosure provides a biological information extraction device including: a center information communication unit configured to communicate with a terminal information communication unit configured to communicate position information of a user and biological information of a user to outside, and receive the position information and the biological information; an accumulation unit configured to accumulate, based on the position information received by the center information communication unit, the number of users having a same value of the biological information of the position; and an accumulation information selection unit configured to select biological information corresponding to a largest accumulation number of users for each position.

In this configuration, the biological information extraction device acquires the position information of the user and the biological information of the user, accumulates the biological information of the users for each position of the user, selects the biological information corresponding to the largest accumulation number of users for each position.

Accordingly, the biological information extraction device can allow the content provider to easily confirm the biological information of the users for each position.

In an embodiment of the present disclosure, the biological information extraction device further includes a display data control unit configured to acquire map information and generate display data in which biological information of majority of the users and the map information are associated with each other.

In this configuration, the biological information extraction device acquires the map information and generates the display data in which the biological information of the majority of users and the map information are associated with each other.

Accordingly, the biological information extraction device can display the biological information of the majority of users on the map. As a result, the content provider can easily visually recognize the biological information of the user and the position information of the user.

Although the embodiments are described above with reference to the drawings, it is needless to say that the present disclosure is not limited to such examples. It will be apparent to those skilled in the art that various changes and modifications may be conceived within the scope of the claims. It is also understood that the various changes and modifications belong to the technical scope of the present invention.

The present application is based on Japanese Patent Application No. 2020-090695 filed on May 25, 2020, and the contents thereof are incorporated herein by reference.

### Industrial Applicability

According to the present disclosure, it is useful as an event information evaluation device, a biological information extraction system, an event information evaluation system, and a biological information extraction device for evaluating a content provided by a content provision based on biological information of a user.

### Reference Signs List

TM1 to TMn: information terminal

CT: center device

100: biological information extraction system

101: position information acquisition unit

102: image capturing unit

103: microphone

104: biological information extraction unit

105: terminal information communication unit

106: center information communication unit

107: emotion information analyzing unit

108: information control device

109: accumulation unit

110: accumulation information selection unit

111: display data control unit

112: map information

113: display unit

114: content execution application

115: event information providing unit

1. An event information evaluation device capable of communicating with a plurality of terminals, each of the plurality of terminals being configured to acquire position information of a user and biological information of the user, the event information evaluation device comprising:
a first processor; and
a first memory storing instructions that, when executed by the first processor, cause the event information evaluation device to perform first operations, the first operations comprising:
acquiring the position information and the biological information from at least one of the plurality of terminals;
based on position information, a number of users having a same value of the biological information corresponding to the position information;
providing event information to the user; and
selecting biological information corresponding to a largest accumulation number of users for each piece of the position information and associating the event information with the biological information corresponding to the largest accumulation number of users.

2. The event information evaluation device according to claim 1, wherein the first operations further comprise:
analyzing the biological information and estimating emotion information of the user.

3. A biological information extraction system comprising:
a plurality of terminals; and
an information control device,
wherein each of the plurality of terminals comprises:
a first processor; and
a first memory storing instructions that, when executed by the first processor, cause a corresponding one of the plurality of terminals to perform first operations, the first operations comprising:
acquiring position information of a user;
acquiring biological information of the user; and
transmitting the position information and the biological information to the information control device, and
wherein the information control device comprises:
a second processor; and
a second memory storing instructions that, when executed by the second processor, cause the information control device to perform operations, the second operations comprising:
acquiring the position information and the biological information from at least one of the plurality of terminals;
accumulating a number of users having a same value of the biological information corresponding to the position information; and
selecting biological information corresponding to a largest accumulation number of users for each piece of the position information.

4. The biological information extraction system according to claim 3,
wherein the biological information corresponding to the largest accumulation number of users is biological information of majority of the users for each piece of the position information.

5. The biological information extraction system according to claim 3, wherein the second operations further comprise:
analyzing the biological information and estimating emotion information of the user.

6. The biological information extraction system according to claim 3, wherein the second operations further comprise:
acquiring map information and generating display data in which biological information of majority of the users and the map information are associated with each other.

7. An event information evaluation system comprising:
a plurality of terminals; and
an information control device comprising the event information evaluation device according to claim 1,
wherein each of the plurality of terminals comprises:
a second processor; and
a second memory storing instructions that, when executed by the second processor, cause a corresponding one of the plurality of terminals to perform second operations, the second operations comprising:
acquiring position information of a user;
acquiring biological information of the user; and
transmitting the position information and the biological information to the information control device.

8. The event information evaluation system according to claim 7,
wherein the biological information corresponding to the largest accumulation number of users is biological information of majority of the users for each piece of the position information.

9. The event information evaluation system according to claim 7, wherein the first operations further comprise:
analyzing the biological information and estimating emotion information of the user.

10. A biological information extraction device capable of communicating with a plurality of terminals, each of the plurality of terminals being configured to acquire position information of a user and biological information of the user, the biological information extraction device comprising:
a processor; and
a memory storing instructions that, when executed by the processor, cause the biological information extraction device to perform operations, the operations comprising:
acquiring the position information and the biological information from at least one of the plurality of terminals;
accumulating a number of users having a same value of the biological information corresponding to the position information, and
selecting biological information corresponding to a largest accumulation number of users for each piece of the position information.

11. The biological information extraction device according to claim 10, wherein the operations further comprise:
acquiring map information and generating display data in which biological information of majority of the users and the map information are associated with each other.
